(19) United States
(12) Patent Application Publication
(10) Pub. No.: US 2025/0320513 A1
(43) Pub. Date: Oct. 16, 2025

(54) METHOD FOR REGULATING AND CONTROLLING PLANT DISEASE AND INSECT PEST RESISTANCE BY HPS1 GENE

(71) Applicant: Sichuan Agricultural University, Chengdu City (CN)

(72) Inventors: Xuewei CHEN, Chengdu City (CN); Haicheng LIAO, Chengdu City (CN); Yu FANG, Chengdu City (CN); Yingjie WEI, Chengdu City (CN); Jiankui CHA, Chengdu City (CN); Jing WANG, Chengdu City (CN); Min HE, Chengdu City (CN); Weitao LI, Chengdu City (CN); Junjie YIN, Chengdu City (CN); Xiaobo ZHU, Chengdu City (CN)

(21) Appl. No.: 19/250,894

(22) Filed: Jun. 26, 2025

Related U.S. Application Data
(63) Continuation of application No. PCT/CN2025/076129, filed on Feb. 7, 2025.

(30) Foreign Application Priority Data
Feb. 26, 2024 (CN) 202410207266.7

(51) Int. Cl. C12N 15/82

(52) U.S. Cl. CPC C12N 15/8205; C12N 15/8281; C12N 15/8282; C12N 15/8286

(57) ABSTRACT

The disclosure relates to a method for regulating and controlling disease resistance in a rice plant by reducing disease resistance of the plant by knocking out the HPS1 gene or protein and/or improving disease resistance of the plant by overexpressing the HPS1 gene or protein. Compared with wild-type rice plants, rice overexpressing HPS1 gene has stronger resistance to different fungal or bacterial diseases, such as rice blast, sheath blight and bacterial blight, and has stronger resistance to insect pests caused by brown planthopper. The knockout of the HPS1 gene leads to a disruption in its protein-encoding ability, thereby causing the rice to exhibit weaker resistance to biological stress compared to wild-type rice.

### CROSS-REFERENCE TO RELATED APPLICATIONS

This application is a continuation of International Patent Application No. PCT/CN2025/076129, filed on Feb. 7, 2025, and claims priority of Chinese Patent Application No. 202410207266.7, filed on Feb. 26, 2024. The entire contents of International Patent Application No. PCT/CN2025/076129 and Chinese Patent Application No. 202410207266.7 are incorporated herein by reference.

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:
File name: SequenceListing.xml
Creation date: Jun. 26, 2025
Byte size: 19,916

### TECHNICAL FIELD

The disclosure relates to the field of crop breeding, in particular to a method for regulating and controlling plant disease and insect pest resistance by HPS1 (Hydrogen peroxide sensor 1) gene or protein encoded by HPS1 gene.

### BACKGROUND

Rice is one of the most important food crops in the world, and it is the staple food for more than half of the population. Rice is also the largest grain crop in China, so its yield is related to China's food security. Biological stress seriously threatens the safe production of rice, especially diseases and pests. At present, chemical control is the main way to control rice diseases and pests, but the chemical control often treats the symptoms rather than the root cause, which will not only cause drug resistance of pathogenic bacteria and pests, but also bring a series of worrying problems such as environmental pollution, ecological crisis, food security and health hazards.

Therefore, it is the most effective and green means to improve crops by excavating and utilizing the genes and mechanisms of resistance to biological stress. Transcription factors are a kind of protein with DNA binding domain, which can bind specific cis-acting elements in gene promoter region to regulate the expression of target genes, and widely participate in the regulation and control of plant resistance to biological stress. At least 167 bHLH family transcription factor genes have been identified in rice, but there are few reports on the function of these transcription factors in the regulation and control of biological stress resistance. Therefore, it is of great importance to identify the bHLH transcription factor that can regulate and control the biological stress resistance and use this factor to improve the biological stress resistance of crops.

### SUMMARY

The purpose of the present disclosure is to provide a method for regulating and controlling plant disease and insect pest resistance by knocking out the HPS1 gene and/or improving disease resistance of the plant by overexpressing the HPS1 gene, so as to solve the problems existing in the prior art. According to the disclosure, HPS1 gene for regulating and controlling the plant disease and insect pest resistance is discovered for the first time, and the plant disease and insect pest resistance can be improved by overexpressing the gene.

To achieve the above objectives, the present disclosure provides the following scheme.

The disclosure provides a method for regulating and controlling plant disease resistance by knocking out the HPS1 gene and/or improving disease resistance of the plant by overexpressing the HPS1 gene, where a nucleotide sequence of the HPS1 gene is shown in SEQ ID NO.1; and an amino acid sequence of the protein is shown in SEQ ID NO.2.

Optionally, the regulating and controlling include improving plant disease resistance or reducing plant disease resistance.

Optionally, reducing the plant disease resistance by knocking out the HPS1 gene; and improving the plant disease resistance by overexpressing the HPS1 gene.

Optionally, the disease resistance includes an ability to resist diseases caused by fungi and an ability to resist diseases caused by bacteria.

Optionally, the fungi include pathogens causing rice blast and/or pathogens causing sheath blight.

The bacteria include pathogens causing bacterial blight.

Optionally, the plant includes rice.

The disclosure provides a method for regulating and controlling plant insect pest resistance, where a nucleotide sequence of the HPS1 gene is shown in SEQ ID NO.1; and an amino acid sequence of the protein is shown in SEQ ID NO.2.

Optionally, the regulating and controlling include improving the plant insect pest resistance and reducing the plant insect pest resistance.

Optionally, reducing insect pest resistance of a plant by knocking out the HPS1 gene; and improving the insect pest resistance of the plant by overexpressing the HPS1 gene.

Optionally, the plant includes rice.

The disclosure discloses the following technical effects.

The disclosure provides a method for regulating and controlling the plant disease by knocking out the HPS1 gene and/or improving disease resistance of the plant by overexpressing the HPS1 gene and insect pest resistance. According to the disclosure, a series of means such as genetics, molecular biology, pathology and the like are utilized to comprehensively identify the resistance function of rice transcription factor gene HPS1 to rice biological stress. According to the disclosure, compared with wild-type rice plants, rice overexpressing HPS1 gene has stronger resistance to different fungal or bacterial diseases, such as rice blast, sheath blight and bacterial blight, and has stronger resistance to insect pests caused by brown planthopper. Knocking out of HPS1 gene results in that the rice whose ability of HPS1 gene to encode protein is hindered has weaker resistance to biological stress than wild-type rice. Therefore, the disclosure identifies an important gene for regulating and controlling rice resistance to various biological stresses in rice, and the HPS1 gene provides a significant theoretical basis for breeding crop varieties with high resistance to biological stresses.

In a specific embodiment, the disclosure successfully obtains two independent knockout strains of HPS1 gene function loss through gene editing. The overexpression plasmid is transformed into rice by Agrobacterium tumefaciens, and two independent overexpression rice strains of HPS1 gene are successfully obtained. At the same time, the overexpression HPS1 gene strain is obtained. According to the phenotypic identification of these transgenic strains, the disclosure explores the regulation and control effect of HPS1 gene on crop biological stress resistance. The regulation and control effect of HPS1 gene on crop biological stress resistance is that overexpression of HPS1 gene can enhance the resistance of rice to different fungal or bacterial diseases such as rice blast, sheath blight and bacterial blight, and enhance the resistance to insect pests caused by brown planthopper, while after HPS1 gene is knocked out, the resistance of rice to biological stresses such as these diseases and insect pests is weakened. Therefore, HPS1 gene can be used to improve the biological stress resistance of different crops.

### BRIEF DESCRIPTION OF THE DRAWINGS

To explain the embodiments of the present disclosure or the technical scheme in the prior art more clearly, the drawings needed in the embodiments will be briefly introduced below. Obviously, the drawings described below are only some embodiments of the present disclosure, and other drawings can be obtained according to these drawings without creative work for ordinary people in the field.

FIG. 1A is a schematic diagram of two independent targets designed by knocking out HPS1 gene with CRISPR/Cas9 system.

FIG. 1B is a comparison diagram of the knock-out target sequences in two independent HPS1-KO transgenic strains (HPS1-KO #1 and HPS1-KO #6) and Kitaake plants, and the two independent HPS1-KO transgenic strains were obtained by PCR sequencing.

FIG. 1C is a comparison diagram of predicted amino acid sequences of HPS1 protein in Kitaake, HPS1-KO #1 and HPS1-KO #6 plants.

FIG. 1D shows the gene expression of HPS1 gene in Kitaake and different HPS1-OE plants (average±s.d., n=3 samples); the data are analyzed by two-tailed student t-test; and the specific P value has been shown in the figure, with P<0.05 as significant difference and P<0.01 as extremely significant difference.

FIG. 1E shows the accumulation of HPS1 protein in HPS1-OE plants by western blot analysis, and the HPS1-YFP protein indicated by arrows is obtained by GFP-trap agarose Immunoprecipitation (IP) of extracted total protein of rice leaves.

FIG. 2A shows that leaves of three-week-old Kitaake, HPS1-KO and HPS1-OE plants are performed stab inoculation with Magnaporthe grisea (physiological race Zhong10-8-14), showing the photos of representative lesions and the statistics (average±s.d., n=9 lesions) of lesion length of rice leaves 7 days after inoculation, and the scale is 1 centimeter (cm).

FIG. 2B shows that the resistance of rice to Magnaporthe grisea is enhanced by HPS1 gene in the field; the three-week-old Kitaake, HPS1-KO and HPS1-OE plants are sprayed with Magnaporthe grisea (physiological race Zhong10-8-14) in the field for spray inoculation; the figure shows the photos of typical lesions of rice leaves after seven days of spray inoculation and the number of lesions on each leaf (average±s.d., n≥7 leaves); the data are analyzed by two-tailed student t-test, and the specific P value is shown in the figure, with P<0.05 as significant difference and P<0.01 as extremely significant difference, and the scale is 2 cm.

FIG. 3 is the identification of the function of HPS1 gene in regulating and controlling rice resistance to sheath blight, which shows typical lesion photos of leaves of three-week-old Kitaake, HPS1-KO and HPS1-OE plants inoculated with Rhizoctonia solani (physiological race AG-1-IA) for two days and statistics of lesion length (average±s.d., n=10 lesions); the scale is 1 cm, and the data are analyzed by two-tailed student t-test; and the specific P value has been shown in the figure, with P<0.05 as significant difference and P<0.01 as extremely significant difference.

FIG. 4 is the identification of the function of HPS1 gene in regulating and controlling rice resistance to bacterial blight, which shows that leaves of three-week-old Kitaake, HPS1-KO and HPS1-OE plants are performed inoculation with Xanthomonas oryzae PV. oryzae (physiological race PXO99A), showing the photos of representative disease spots and the statistics of disease spot length (average SD, n=10 disease spots) 14 days after inoculation; the data are analyzed by two-tailed student t-test; the specific P value has been shown in the figure, with P<0.05 as significant difference and P<0.01 as extremely significant difference; and the scale is 1 cm.

FIG. 5 is the identification of the function of HPS1 gene in regulating and controlling rice resistance to brown planthopper; the figure shows the phenotypes and BPH resistance scores (average±SD, n=30 samples) of Kitaake, HPS1-KO and HPS1-OE seedlings in the two-leaf stage after being sucked by brown planthopper for 4 days; the data are analyzed by two-tailed student t-test; and the specific P value has been shown in the figure, with P<0.05 as significant difference and P<0.01 as extremely significant difference.

FIG. 6 is a working schematic diagram of improving rice resistance to biological stress by using HPS1 gene.

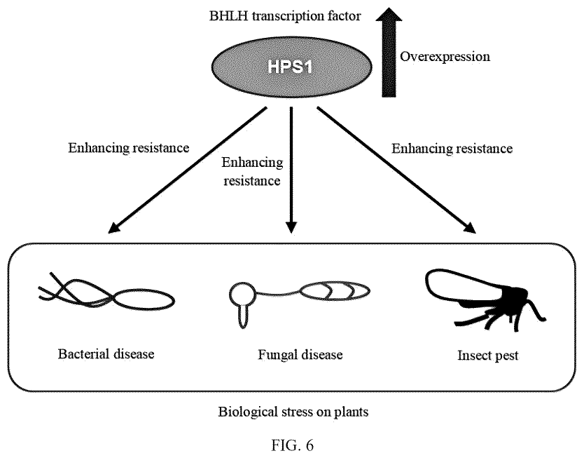

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges can be independently included or excluded from the range.

Unless otherwise specified, all technical and scientific terms used herein have the same meaning as commonly understood by one of ordinary skill in the art to which this disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes can be made to the specific embodiments of the present disclosure without departing from the scope or spirit of the present disclosure. Other embodiments will be apparent to the skilled person from the description of the disclosure. The description and embodiment of the present disclosure are exemplary only.

The terms “comprising”, “including”, “having” and “containing” used in this article are all open terms, which means including but not limited to.

The commercially available pCRISPR and pCAMBIA1300 vectors used in the following embodiments, and the published Magnaporthe grisea (physiological race Zhong10-8-14), Rhizoctonia solani (physiological race AG-1-IA), Xanthomonas oryzae PV. oryzae (physiological race PXO99A), brown planthopper (collected in the greenhouse of Sichuan Agricultural University in 2021) and rice materials (wild-type Kitaake, HPS1-KO and HPS1-OE) are provided and preserved by the State Key Laboratory of Southwest Genetic Resources Exploration and Utilization of Sichuan Agricultural University.

### Embodiment 1 Construction of Mutant Rice Plants with HPS1 Gene Knocked Out

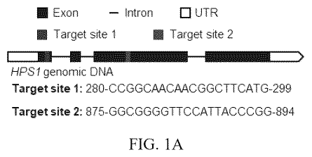

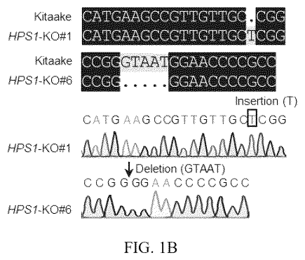

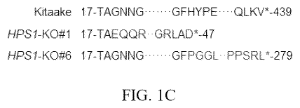

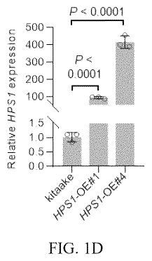

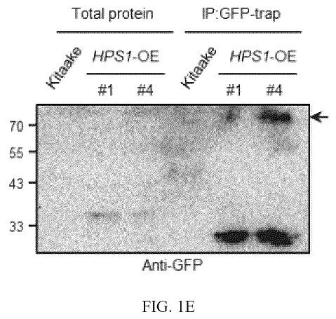

In order to identify the function of HPS1 gene (ID: Os01g0196300), the inventor uses CRISPR/Cas9 system to design two knockout targets (Target sites1 and Target sites2) for the HPS1 gene (SEQ ID NO.1) in wild-type Kitaake plants for gene editing. The nucleotide sequence of Target sites1 is shown in SEQ ID NO.13, specifically CCGGCAACAACGGCTTCATG, and the nucleotide sequence of Target sites2 is shown in SEQ ID NO.14, specifically GGCGGGGTTCCATTACCCGG (FIG. 1A).

Genomic sequence of HPS1 gene in rice (SEQ ID NO.1):

>NC_029256.1:5201837-5203986 Oryza sativa Japonica Group cultivar Nipponbare chromosome 1, IRGSP-1.0:

The inventors synthesizes the designed target sequence into primers, and anneals the “pCRISPR-HPS1-Target1-KO” primer and “pCRISPR-HPS1-Target2-KO” primer (Table 1) in a PCR instrument (95 degrees Celsius (C) for 3 minutes (min), 0.2 degrees Celsius per second (° C./sec) to 20° C.). Then, the pCRISPR plasmid is ligated with the annealed primer by vector exonuclease linearization-ligation reaction system (Table 2). Agrobacterium carrying the constructed knockout vector is transformed into the callus of wild-type rice Kitaake to obtain transgenic rice plants (methods refer to W. Li, et al., A Natural Allele of a Transcription Factor in Rice Confers Broad-Spectrum Blast Resistance. Cell 170(1): 14-126, 2017).

TABLE 1
Primer sequences related to detection of HPS1 knockout plants

| | | | SEQ ID | |
|---|---|---|---|---|
| Primer name | Primer sequence (5′-3′) | | NO. | Use |
| pCRISPR- | Forward | GCAGGTCTCATGTGCCGGCAACAAC | 5 | Construction |
| HPS1-Target1- | primer | GGCTTCATGGTTTTAGAGCTAGAAA | | of a Vector |
| KO | | TAGCAAGTT | | Knocking out |
| | Reverse | GCAGGTCTCTAAAACCATGAAGCCG | 6 | target 1 in |
| | primer | TTGTTGCCGGTGCCACGGATCATCT | | HPS1 Gene |
| | | GCA | | |
| pCRISPR- | Forward | GCAGGTCTCATGTGCCGGCAACAAC | 7 | Construction |
| HPS1-Target2- | primer | GGCTTCATGGTTTTAGAGCTAGAAA | | of a Vector |
| KO | | TAGCAAGTT | | Knocking out |
| | Reverse | GCAGGTCTCTAAAACCATGAAGCCG | 8 | target 2 in |
| | primer | TTGTTGCCGGTGCCACGGATCATCT | | HPS1 Gene |
| | | GCA | | |
| HPS1-KO- | Forward | CTTCTGCGGCTTTCATTGGC | 9 | Detection of |
| target-1- | primer | | | target 1 |
| detection | Reverse | CAGCCAAACCACTCAAGAACA | 10 | knockout |
| | primer | | | situation in |
| | | | | HPS1 gene |
| | | | | knockout |
| | | | | plants |
| HPS1-KO- | Forward | GTGTCGCGTTTTATTACGGCA | 11 | Detection of |
| target-2- | primer | | | target 2 |
| detection | Reverse | TGTATGTATGTATACCTTAGAGCCG | 12 | knockout |
| | primer | | | situation in |
| | | | | HPS1 gene |
| | | | | knockout |
| | | | | plants |

TABLE 2
Vector exonuclease linearization-ligation reaction system

| Component | Dosage | |
|---|---|---|
| pCRISPR plasmid | 100 nanograms | |
| | (ng) | |
| Annealing primer | 10 | ng |
| BsaI exonuclease | 0.5 microliters | |
| | (μL) | |
| T4 ligase | 2.5 | μL |
| Anza buffer | 1 | μL |

| | |
|---|---|
| Total | To 5 μL |

Next, the inventors uses the PCR reaction (Table 3 and Table 4) and the primer “HPS1-KO-target-1-detection” (Table 1) to detect the change of Target 1 in transgenic plants, and uses the PCR reaction and the primer “HPS1-KO-target-2-detection” (Table 1) to detect the change of Target 2 in transgenic plants. The results show that HPS1 knockout (HPS1-KO) #1 strain inserts a T base in target 1, while another HPS1-KO #6 strain has a deletion of GTAAT base in target 1 (FIG. 1B). Therefore, the above experiments show that the inventors have successfully obtained rice plants with HPS1 gene knocked out.

TABLE 3
PCR reaction system

| Component | Dosage | |
|---|---|---|
| 10× PCR Buffer | 2.5 | μL |
| MgSO₄ (2 millimolars (mM)) | 1.5 | μL |
| dNTP (2.5 mM each) | 2.5 | μL |
| 50% glycerol | 2.5 | μL |
| Forward primer (10 micromolars | 0.2 | μL |
| (μM)) | | |
| Reverse primer (10 μM) | 0.2 | μL |
| KOD-Plus Polymerase | 0.5 | μL |
| Genome | 1.0 | μL |
| ddH₂O | 14.1 | μL |
| Total | 25 | μL |

After the above samples are mixed evenly and centrifuged, PCR reaction is carried out, and PCR amplification is carried out by touch-down PCR. The reaction procedure is shown in Table 4.

TABLE 4
PCR reaction procedure

| | | |
|---|---|---|
| Temperature | Time | Cycle |

| 95° | C. | 5 | min | |
|---|---|---|---|---|
| 95° | C. | 30 | seconds (sec) | |
| 58° | C. | 30 | sec | 28-34 cycles |
| 68° | C. | 30 sec(~1 | | |
| | | kilobase pair per | | |
| | | minute (kb/min)) | | |
| 68° | C. | 10 | min | |

| | | |
|---|---|---|
| 4° | C. | Preservation |

Further analysis shows that each mutation in HPS1-KO #1 and HPS1-KO #6 plants will lead to frame shift and premature termination of HPS1 protein (SEQ ID NO.2-4 and FIG. 1C). Therefore, the above results indicate that the inventors have successfully obtained two independent knockout strains with loss of HPS1 gene function.

The amino acid sequence of HPS1 protein in rice (SEQ ID NO.2):

HPS1 protein amino acid sequence in HPS1-KO #1 rice after gene editing (SEQ ID NO.3):

MEDCSSWIHGYANANATAEQQRLHVRLRCQLQPSRVSAAATAGRLAD*,

where “*” is the meaning of stop codon.

HPS1 protein amino acid sequence in HPS1-KO #6 rice after gene editing (SEQ ID NO.4):

MEDCSSWIHGYANANATAGNNGFMCGYAASCSPVEFQQQQQLVGSQIEH
HLNQISMQMGMDDESAVYDGASMVDVLLMASSSPHHHAGAGSFQYSSPT
SSSASFRSASVSCSPESSAAATTHELGPPAPSAAAAGFPGGLLAGAVAT
TLAALRAAARPIHHRPLAAAAGARVAGDYYAGDRRRVQAVRAAPPPEEA
AQAGRVRAEDVQDGHVGAHQDARGGDVQPPVLLPAGGSRRRVGVGGRGA
AVRQPAAAHDLGAEAAGEAQRQLPRPQGRPPSRL*,

where “*” means the stop codon.

### Embodiment 2 Construction of Rice Plants Overexpressing HPS1 Gene

The inventor amplifies the coding region sequence of HPS1 by PCR reaction (Table 3 and Table 4) with the primer “pCAMBIA1300-35S:HPS1-OE” (Table 5). Then pCAMBIA1300-35S:HPS1 plasmid is constructed by in vitro recombination method (Table 6), and is transformed into wild-type Kitaake plants by Agrobacterium tumefaciens to obtain several transgenic strains. Subsequently, the total RNA of rice is extracted by Trizol method, and the specific experimental steps are as follows: the mortar is cleaned, dried, poured alcohol and ignited, and the RNAase in the mortar is removed at high temperature. Freezing the collected samples with liquid nitrogen, grinding them, putting them into a tube, and adding an appropriate amount of Trizol reagent (in principle, 1 milliliter (mL) of Trizol is added to every 100 milligrams (mg) of samples, and the following data are calculated according to 100 mg of samples). After the Trizol solution and the sample powder are fully mixed, they are left at room temperature for 5 min. Adding 0.2 mL chloroform, vortex violently for 15 sec, and standing at room temperature for 3 min. Putting the sample into a centrifuge, centrifuging at 4° C. for 15 min at 12000 revolutions per minute (r/min), and carefully transferring the upper water phase to a new EP tube (about 0.4 mL). Adding chloroform with the same volume, vortex violently for 15 sec, centrifuging at 4° C. for 15 min at 12000 r/min, and transferring the upper water phase to a new EP tube (about 0.2 mL) again. Adding isopropanol, standing at room temperature for 10 min, and centrifuging at 4° C. for 10 min at 12000 r/min. Removing the supernatant, adding 0.75 mL of 70% ethanol, flicking the bottom of the tube by hand, centrifuging at 4° C. for 5 min at 10000 r/min, and removing the supernatant. Putting the EP tube in a fume hood, and adding 30 μL RNAase free water to fully dissolve RNA after the alcohol is completely volatilized. Reverse transcription synthesizes the first strand of cDNA according to the operating instructions of SuperScript™ III Reverse Transcriptase kit of Thermo scientific Company. (see Table 7 and Table 8). Then, the quantitative PCR system is configured by using the “qHPS1” primer in Table 5 and the synthesized cDNA (method check Table 9). After the system is added, it is placed on CFX96TM Real-Time System (Bio-Rad, USA) for PCR reaction. The reaction condition is two-step PCR, and the procedure is as follows: 95° C./30 sec; 95° C./5 sec, 58° C./30 sec, with 39 cycles. And then the temperature is increased by 0.5° C. per second until 95° C., thereby generating a dissolution curve. When analyzing the quantitative results, the 2^{-ΔΔCt} method of CFX96™ Real-Time System instrument is used to calculate. Results as shown in FIG. 1D, the expression of HPS1 gene in two HPS1 overexpression (HPS1-OE) strains HPS1-OE #1 and HPS1-OE #4 is significantly higher than the expression of HPS1 gene in wild type.

TABLE 5
Primer sequences related to construction of plants with overexpression of HPS1
gene

| Primer | | | SEQ ID | |
|---|---|---|---|---|
| name | Primer sequence (5′-3′) | | NO. | Use |
| pCAMBIA | Forward | AGAACACGGGGGACGAG | 15 | Construction of plants |
| 1300-35S: | primer | CTCATGGAGGATTGCAGC | | with overexpression |
| HPS1-OE | | AGCTG | | of HPS1 gene |
| | Reverse | CCCTTGCTCACCATGGTAC | 16 | |
| | primer | CCACCTTGAGCTGTAATGT | | |
| | | TA | | |
| qHPS1 | Forward | GGCAACAACGGCTTCATG | 17 | Detection of HPS1 |
| | primer | TG | | gene expression in |
| | Reverse | TGAGGTGGTGCTCAATCT | 18 | plants with |
| | primer | GC | | overexpression of |
| | | | | HPS1 gene |

TABLE 6
Vector recombination in vitro reaction system

| Component | Dosage | |
|---|---|---|
| 5× CE Buffer | 2 | μL |
| Enzyme digestion vector | 2 | μL |
| Target fragment | 2 | μL |
| Recombinant enzyme in vitro | 1 | μL |
| ddH₂O | 3 | μL |
| Total | 10 | μL |

The ingredients in Table 7 are added to the DEPC-treated PCR tubes in turn.

TABLE 7
Reverse transcription reaction system-1

| Component | Dosage | |
|---|---|---|
| Oligo dT | 1.0 | μL |
| RNA | 2.0 | μL |
| 10 mM dNTP Mix | 1.0 | μL |
| ddH2O | 9.0 | μL |
| Total | 13.0 | μL |

After slight mixing, centrifuging, heating at 65° C. for 5 min, placing on ice for 3 min, and then adding the ingredients in Table 8.

TABLE 8
Reverse transcription reaction system-2

| Component | Dosage | |
|---|---|---|
| 5X First-Strand Buffer | 4.0 | μL |
| 0.1M DTT | 1.0 | μL |
| RNase Inhibitor | 1.0 | μL |
| SuperScript ™ III RT | 1.0 | μL |
| Total | 7.0 | μL |

Reacting at 50° C. for 60 min; and the reaction is terminated at 70° C. for 15 min and preserved at −20° C.

TABLE 9
Quantitative PCR reaction system

| Component | Dosage | |
|---|---|---|
| Primer | 1 | μL |
| cDNA | 1 | μL |
| KAPA SYBR ® Premix | 5 | μL |
| ddH₂O | 3 | μL |
| Total | 10 | μL |

In order to detect whether the content of HPS1 protein in HPS1-overexpressing plants has changed, the inventor conducts the detection experiment of Immunoprecipitation (IP) combined with western blot of HPS1 protein on wild-type Kitaake plants and HPS1-overexpressing plants (HPS1-OE #1 and HPS1-OE #4). The specific steps are as follows: freezing and grinding the leaves to powder, then taking out the precooled 50 mL low-speed centrifugal tube, weighing the centrifugal tube, peeling it, and then adding the powder into the centrifugal tube for weighing. Adding IP buffer (50 mM HEPES [pH 7.5], 150 mM KCl, 1 mM EDTA, 0.5% Trition-X 100, 1 mM DTT, 1 mM protease inhibitor cocktail) according to 1 gram (g)/2 mL, and letting it stand at room temperature until the ice completely turns into water. Putting the centrifuge tube on ice for 30 min, and shaking it every 10 min. During the incubation period, taking another 1.5 mL centrifuge tube, adding 50 μL anti-GFP magnetic beads to 1 mL IP buffer, washing once, putting it on a magnetic rack, sucking away the supernatant, adding 0.5 mL IP buffer and putting it on ice. Sucking the incubated liquid into a 1.5 mL centrifuge tube, centrifuging at 12300 revolutions per minute (rpm) at 4° C. for 40 min, and sucking the centrifuged supernatant into a 10 mL centrifuge tube, taking 60 μL supernatant as a test sample, then adjusting the supernatant of each centrifuge tube to the same volume, resuspending the previous anti-GFP magnetic beads with 200 μL IP buffer and mixing them evenly. After adding 200 μL GFP magnetic beads to each centrifuge tube, rotating vertically at 4° C. for at least 4 hours (h), adding the incubated liquid into the 1.5 mL centrifuge tube on the magnetic rack, and sucking out the supernatant until the liquid is completely added. Washing with IP buffer for 7-8 times, finally adding 40 μL IP buffer as IP, adding loading buffer, heating anti-GFP magnetic beads, and spotting it for western blot detection. The experimental results show that, as shown in FIG. 1E, compared with the wild-type Kitaake plant, more HPS1 protein is enriched in HPS1-OE #1 strain and HPS1-OE #4 strain. Therefore, the above results indicate that there is obvious HPS1 protein accumulation in two independent overexpression strains of HPS1 gene.

### Embodiment 3 Identification of the Role of HPS1 Gene in Regulating and Controlling Rice Resistance to Rice Blast

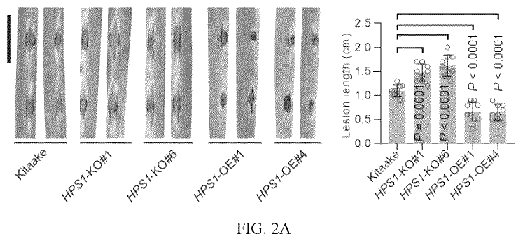

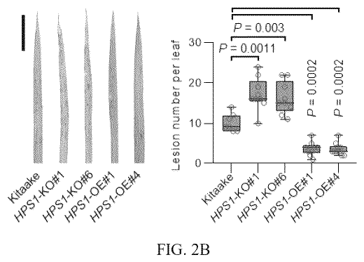

In order to test whether the HPS1 gene regulates and controls the resistance of rice to rice blast, the inventors firstly stabs the leaves of rice seedlings of three-week-old wild Kitaake, HPS1-KO (HPS1-KO #1 and HPS1-KO #6) and HPS1-OE (HPS1-OE #1 and HPS1-OE #4), and inoculates 5 μL of spore suspension (5×10⁵ spores/mL) of Magnaporthe grisea (physiological race Zhong10-8-14), and analyzing statistically the size of the lesion after incubation at 28° C. for 7 days. The results of stab inoculation are shown in FIG. 2A. Compared with wild-type Kitaake, the lesion on the leaves of HPS1-KO plants is larger, while the lesion on the leaves of HPS1-OE plants is smaller.

In the spray inoculation experiment, the inventor sprays the spore suspension of Magnaporthe grisea (5×10⁵ spores/mL) on the leaves of three-week-old seedlings, and counts the number of lesions on each infected leaf in the field after 7 days. The experimental results are shown in FIG. 2B. Compared with wild-type Kitaake, the lesions on the leaves of HPS1-KO plants are larger, while the lesions on the leaves of HPS1-OE plants are smaller. Therefore, the above results indicate that HPS1 gene can positively regulate and control rice resistance to rice blast.

### Embodiment 4 Identification of the Role of HPS1 Gene in Regulating and Controlling Rice Resistance to Sheath Blight

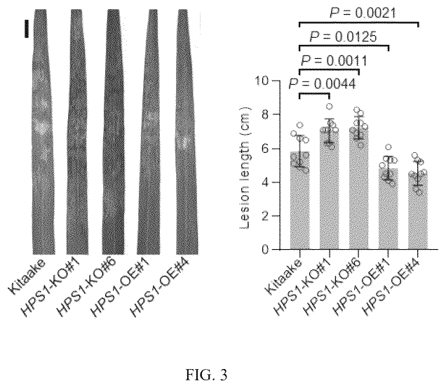

In order to test whether the HPS1 gene regulates and controls the resistance of rice to sheath blight, the inventor places the mycelium blocks (culturing on PDA medium for 2 days) of physiological race AG-1-IA strain of Rhizoctonia solani with uniform size on rice leaves of Kitaake, HPS1-KO (HPS1-KO #1 and HPS1-KO #6) and HPS1-OE (HPS1-OE #1 and HPS1-OE #4) plants at tillering stage, and analyzing statistically the size of the lesion after incubating at 28° C. for 2 days. The experimental results are shown in FIG. 3. Compared with wild-type Kitaake, the lesion on the leaves of HPS1-KO plant is larger, while the lesion on the leaves of HPS1-OE plant is smaller. Therefore, the above results indicate that HPS1 gene can positively regulate and control the resistance of rice to sheath blight.

### Embodiment 5 Identification of the Role of HPS1 Gene in Regulating and Controlling Rice Resistance to Bacterial Blight

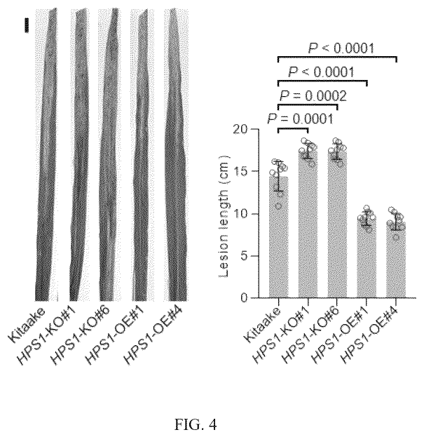

In order to test whether HPS1 regulates and controls the resistance of rice to bacterial blight, the inventor cuts off rice leaves of Kitaake, HPS1-KO (HPS1-KO #1 and HPS1-KO #6) and HPS1-OE (HPS1-OE #1 and HPS1-OE #4) at tillering stage, and cuts off the position about 1 cm away from the tip. Scissors used in the experiment are immersed in the bacterial (cultured on PDA medium for 2 days) suspension of physiological race PXO99A (OD₆₀₀=0.6) of Xanthomonas oryzae PV. oryzae in advance, so the cut leaf wound will be infected with Xanthomonas oryzae PV. oryzae and then get sick, and then the size of the lesion is analyzed statistically after the disease is completely ill. The experimental results are shown in FIG. 4. Compared with wild-type Kitaake, HPS1-KO plants have larger lesions on their leaves, while HPS1-OE plants have smaller lesions on their leaves. Therefore, the above results indicate that HPS1 gene can positively regulate and control the resistance of rice to bacterial blight.

### Embodiment 6 Identification of the Role of HPS1 Gene in Regulating and Controlling Rice Resistance to Insect Pest

Brown planthopper is the pest that causes the greatest damage to rice production and application. Therefore, in order to detect whether the HPS1 gene regulates and controls the resistance of rice to insect pests, the inventors carries out the infection experiment of brown planthopper on transgenic plants related to HPS1.

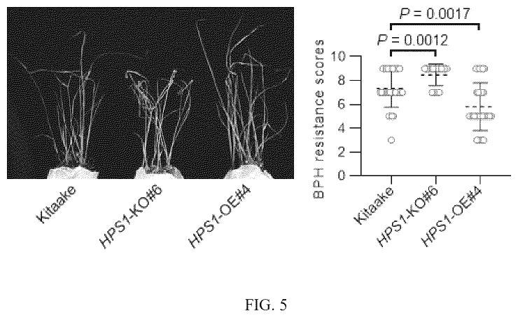

The inventor sows about 15-20 seeds of Kitaake, HPS1-KO (HPS1-KO #1 and HPS1-KO #6) and HPS1-OE (HPS1-OE #1 and HPS1-OE #4) in a plastic cup (about 10 cm in diameter and 20 cm in height), and at the bottom of the cup, the seeds are cultured with cotton that absorbs Yoshida plant nutrient solution (manufacturer: Coolaber, article number: NSP1040). When the seedlings grow to the second leaf stage, the inventor puts the third-year-old brown planthopper nymph into a cup and lets it freely suck the seedlings, and the density of insects is 10 insects each seedling. Surveys are conducted when all susceptible plants die (the score is 9), and each seedling of other varieties or lines is scored as 0, 1, 3, 5, 7 or 9 according to the damage degree (the main reference of this standard is J. Guo, et al., A tripartite rheostat controls self-regulated host plant resistance to insects, Nature, 618(7966): 799-807, 2023). The experimental results are shown in FIG. 5. Compared with wild-type Kitaake plants, the leaf damage degree of HPS1-KO plants is higher, while the leaf damage degree of HPS1-OE plants is lower. Therefore, HPS1 gene can positively regulate and control the resistance of rice to insect pests represented by brown planthopper.

To sum up, the inventors finds that overexpression of HPS1 gene can enhance the resistance of rice to different fungal or bacterial diseases such as rice blast, sheath blight and bacterial blight, and enhance the resistance to insect pests caused by brown planthopper, while knockout of HPS1 gene weakens the resistance of rice to biological stresses such as these diseases and insect pests. Thus, the HPS1 gene described in the present disclosure provides an important theoretical basis for the selection of crop varieties resistant to modified biological stresses.

The above-mentioned embodiments only describe the preferred mode of the disclosure, and do not limit the scope of the disclosure. Under the premise of not departing from the design spirit of the disclosure, various modifications and improvements made by ordinary technicians in the field to the technical scheme of the disclosure shall fall within the protection scope determined by the claims of the disclosure.

What is claimed is:

1. A method for regulating and controlling disease resistance in a rice plant by at least one of:
reducing disease resistance of a plant by knocking out hydrogen peroxide sensor 1 (HPS1) gene or protein, or
improving the disease resistance of the plant by overexpressing the HPS1 gene or the protein;
wherein the method comprises: introducing the HPS1 gene or the protein into Agrobacterium tumefaciens; and transforming the rice plant with the Agrobacterium tumefaciens carrying the HPS1 gene or the protein to obtain an HPS1-overexpressing transgenic rice strain;
wherein the HPS1 gene comprises a nucleotide sequence of SEQ ID NO: 1 and encodes protein having an amino acid sequence of SEQ ID NO: 2; and
fungi are at least one of pathogens causing rice blast or pathogens causing sheath blight; and bacteria are pathogens causing bacterial blight.

2. A method for regulating and controlling insect pest resistance in a rice plant by at least one of:
reducing disease resistance of a plant by knocking out hydrogen peroxide sensor 1 (HPS1) gene or protein, or
improving the disease resistance of the plant by overexpressing the HPS1 gene or the protein;
wherein the method comprises: introducing the HPS1 gene or the protein into Agrobacterium tumefaciens; and transforming the rice plant with the Agrobacterium tumefaciens carrying the HPS1 gene or the protein to obtain an HPS1-overexpressing transgenic rice strain;
wherein the HPS1 gene comprises a nucleotide sequence of SEQ ID NO: 1 and encodes protein having an amino acid sequence of SEQ ID NO: 2; and
an insect pest is brown planthopper.


SEQUENCE LISTING (18 sequences)

SEQ ID NO: 1 - DNA, 2150 bp, synthetic construct
  source 1..2150: mol_type = other DNA
gcagatatgtgctcactgcctagctaccagtcttgcatctctaactatatatacacatacatgtagcatcctgctccatcttaactagctactactagtgatgagcagaatcaacctcatgcagtaatatgctgtgctgatcgaattaatttacacatatatagatagatagatagatagatcgttcgaccaatcgagctagcttgagcaaactgattgacctggtgaagtggtataattaacgagcagatcatggaggattgcagcagctggatccatggctacgccaacgctaacgccaccgccggcaacaacggcttcatgtgcggctacgctgccaggtaggtgttcaaacatcttgatgatcaccgtgacacgatcgatcgattgatcgcgacgacgacgataataccatctgtaaattagtcgtctctgttcttgagtggtttggctgatcagttagttaattagctacttcgtgtttgcagctgcagcccagtagagtttcagcagcagcaacagctggtcggctcgcagattgagcaccacctcaaccaggcaagcttgatttgcaatttattcgatcaagaaacaatgcataaaatcaaagaaattttaggatttaatttggtaggtgtcgcgttttattacggcatgcagatcagcatgcagatggggatggatgacgagtcggcggtgtacgacggcgcctccatggtggacgtcctcctcatggcttcctcgtcgccgcaccaccacgccggcgccggcagcttccagtactcctcgccgacgtcctcctccgcctccttccgctccgcctccgtctcgtgcagccctgagagctcggcggcggcgacgacgcacttcctcggaccgccggcgccgtccgcggcggcggcggggttccattacccggaggtctcctcgcaggcgccgttgccactacccttgccgccctacgagccgcagcacggccaatacaccaccgtcctctcgccgccgccgccggcgccagagttgccggcgactactacgccggcgaccggcggcgcgttcaggcggtacgcgcggcacctccgcccgaggaggctgcccaagccgggagggtgcgggcagaggatgttcaagacggccatgtcggtgctcaccaagatgcacgtggcggcgacgtacaaccgccagtactactaccagcaggcggcagccgccgccgcgtcggcgtcggcggccgaggcgccgccgtccggcaaccagctgcagcacatgatctcggagcggaagcggcgggagaagctcaacgacagcttcctcgccctcaaggccgtcctccctcccggctctaaggtatacatacatacacgtatcaatacgtacatttgtacgtatacacatgcatgatcattcatcaacattttgttcttttgattagtgatcttgactagtaatatttgcttcaatcatttcttcctttttaatggtagaaagacaagacatcgatactgatcagagcaagagagtacgtaaaatctctcgagtcaaagctgtcagagctggaggagaagaaccgggagctagaggcgaggctagccagccgccccgccgccgccgccaagaacgacaaaggcgagacggcggcggcgccagcgccggaagccggcgatgagacgaagcgaaaggacctagtagagatcgaggtgacgacgagcggcggcggcgccggagcggcggatgcggcggcggcggcgggaggagatcaagagacctgcacgctcaacgtagacttgcgcggcggcggaggcggcggaggcatgagcacgacggacgttgtgctccggacgctgcagtgcctgagagagcagatcggcgacggcgccagcctcgtggcgatgagcaccagcgccggctccggcggccgcccgcctcgtgcaaacctaacattacagctcaaggtgtaattaattaataaaaaagcattctactaaagaagttaaaggaaaaatctgtctcacatctcagtgatcagaaaaaaaaaagaagaaaaagggaggtgccatcagtgcaaaatagcatgcatgcatgcaagcacattgttgacagcatgattgtacatttttctctcttaagataatggaaaaccttacacatat

SEQ ID NO: 2 - protein, 439 aa, synthetic construct
MEDCSSWIHGYANANATAGNNGFMCGYAASCSPVEFQQQQQLVGSQIEHHLNQISMQMGMDDESAVYDGASMVDVLLMASSSPHHHAGAGSFQYSSPTSSSASFRSASVSCSPESSAAATTHFLGPPAPSAAAAGFHYPEVSSQAPLPLPLPPYEPQHGQYTTVLSPPPPAPELPATTTPATGGAFRRYARHLRPRRLPKPGGCGQRMFKTAMSVLTKMHVAATYNRQYYYQQAAAAAASASAAEAPPSGNQLQHMISERKRREKLNDSFLALKAVLPPGSKKDKTSILIRAREYVKSLESKLSELEEKNRELEARLASRPAAAAKNDKGETAAAPAPEAGDETKRKDLVEIEVTTSGGGAGAADAAAAAGGDQETCTLNVDLRGGGGGGGMSTTDVVLRTLQCLREQIGDGASLVAMSTSAGSGGRPPRANLTLQLKV

SEQ ID NO: 3 - protein, 47 aa, synthetic construct
MEDCSSWIHGYANANATAEQQRLHVRLRCQLQPSRVSAAATAGRLAD

SEQ ID NO: 4 - protein, 279 aa, synthetic construct
MEDCSSWIHGYANANATAGNNGFMCGYAASCSPVEFQQQQQLVGSQIEHHLNQISMQMGMDDESAVYDGASMVDVLLMASSSPHHHAGAGSFQYSSPTSSSASFRSASVSCSPESSAAATTHFLGPPAPSAAAAGFPGGLLAGAVATTLAALRAAARPIHHRPLAAAAGARVAGDYYAGDRRRVQAVRAAPPPEEAAQAGRVRAEDVQDGHVGAHQDARGGDVQPPVLLPAGGSRRRVGVGGRGAAVRQPAAAHDLGAEAAGEAQRQLPRPQGRPPSRL

SEQ ID NO: 5 - DNA, 59 bp, synthetic construct
  source 1..59: mol_type = other DNA
gcaggtctcatgtgccggcaacaacggcttcatggttttagagctagaaatagcaagtt

SEQ ID NO: 6 - DNA, 53 bp, synthetic construct
  source 1..53: mol_type = other DNA
gcaggtctctaaaaccatgaagccgttgttgccggtgccacggatcatctgca

SEQ ID NO: 7 - DNA, 59 bp, synthetic construct; identical to SEQ ID NO: 5
  source 1..59: mol_type = other DNA

SEQ ID NO: 8 - DNA, 53 bp, synthetic construct; identical to SEQ ID NO: 6
  source 1..53: mol_type = other DNA

SEQ ID NO: 9 - DNA, 20 bp, synthetic construct
  source 1..20: mol_type = other DNA
cttctgcggctttcattggc

SEQ ID NO: 10 - DNA, 21 bp, synthetic construct
  source 1..21: mol_type = other DNA
cagccaaaccactcaagaaca

SEQ ID NO: 11 - DNA, 21 bp, synthetic construct
  source 1..21: mol_type = other DNA
gtgtcgcgttttattacggca

SEQ ID NO: 12 - DNA, 25 bp, synthetic construct
  source 1..25: mol_type = other DNA
tgtatgtatgtataccttagagccg

SEQ ID NO: 13 - DNA, 20 bp, synthetic construct
  source 1..20: mol_type = other DNA
ccggcaacaacggcttcatg

SEQ ID NO: 14 - DNA, 20 bp, synthetic construct
  source 1..20: mol_type = other DNA
ggcggggttccattacccgg

SEQ ID NO: 15 - DNA, 40 bp, synthetic construct
  source 1..40: mol_type = other DNA
agaacacgggggacgagctcatggaggattgcagcagctg

SEQ ID NO: 16 - DNA, 40 bp, synthetic construct
  source 1..40: mol_type = other DNA
cccttgctcaccatggtacccaccttgagctgtaatgtta

SEQ ID NO: 17 - DNA, 20 bp, synthetic construct
  source 1..20: mol_type = other DNA
ggcaacaacggcttcatgtg

SEQ ID NO: 18 - DNA, 20 bp, synthetic construct
  source 1..20: mol_type = other DNA
tgaggtggtgctcaatctgc